(19) United States
(12) Patent Application Publication
(10) Pub. No.: US 2006/0121000 A1
(43) Pub. Date: Jun. 8, 2006

(54) Methods and vectors for controlling gene expression

(75) Inventors: Richard G Vile, Rochester, MN (US); Atique Ahmed, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research, Rochester, MN (US)

(21) Appl. No.: 10/533,613

(22) PCT Filed: Oct. 31, 2003
(86) PCT No.: PCT/US03/34599

§ 371 Date: Jan. 30, 2006

Related U.S. Application Data
(60) Provisional application No. 60/423,244, filed on Nov. 1, 2002.

(51) Int. Cl. A61K 48/00; C12N 15/86

(52) U.S. Cl. 424/93.2; 435/456

(57) ABSTRACT

Viral vectors that include a nucleic acid encoding a therapeutic polypeptide operably linked to a heterologous destabilizing element are described.

### RELATED APPLICATIONS

This application claims the benefit of U.S. Provisional Application Ser. No. 60/423,244, filed Nov. 1, 2002.

### TECHNICAL FIELD

This invention relates to viral vectors, and more particularly to viral vectors that contain destabilizing elements and methods of controlling therapeutic gene expression using such vectors.

### BACKGROUND

In general, tumor-selective gene expression from adenoviral vectors (replication competent or incompetent) has been achieved through transcriptional regulation using selective promoters driving either essential replicative (Rodriguez, R. et al. Cancer Res. 57, 2559-2563 (1997); Hallenbeck, P. L. et al. Hum. Gene Ther. 10, 1721-1733 (1999); and Kurihara, T., et al., J Clin Invest 106, 763-771. (2000)) or therapeutic genes (Siders, W. M., et al., Cancer Res. 56, 5638-5646 (1996); and Blackburn, R. V., et al., Cancer Res. 58, 1358-1362 (1998)). The promoter/enhancers used for these vectors derive from genes whose expression is selectively up-regulated in tumor cells as opposed to normal counterparts. However, it also is clear that tissue specific promoter/enhancer elements inserted into adenoviral genomes are affected by viral enhancers requiring the addition of additional insulator elements, thereby complicating the efficacy of such approaches (Ring, C. J. A., et al. Gene Ther. 3, 1094-1103 (1996)).

### SUMMARY

The invention is based on the incorporation of one or more destabilizing elements into a viral vector that allow enhanced expression of a therapeutic polypeptide in a target cell relative to the expression of the therapeutic polypeptide in a non-target cell into which the vector has been introduced. As used herein, the term “therapeutic polypeptide” refers to any chain of amino acids that can slow cell growth, alter a physiologic function of the cell, or kill the cell. Therapeutic polypeptides can be essential gene products that allow a virus to replicate. As a result, a viral vector or a virus (e.g., a replication competent virus) containing such a vector can be introduced into a mammal such that a certain cell type (e.g., a tumor cell) is targeted more selectively. Furthermore, selectively targeting tumor cells can allow tumor cells to be destroyed more efficiently, while minimizing toxicity to non-target cells.

The destabilizing element can be at least a portion of the 3′ untranslated region (UTR) of the cyclooxygenase 2 (COX-2) gene, which typically destabilizes its cognate mRNA. In cells in which RAS has been activated, however, the cells contain the necessary machinery to stabilize the COX-2 mRNA, allowing for enhanced expression of the COX-2 enzyme. Thus, a viral vector containing a nucleic acid encoding a therapeutic polypeptide operably linked to a destabilizing element such as the COX-2 gene 3′UTR, can be introduced into a mammal having a tumor in which RAS has been activated, resulting in increased levels of the therapeutic polypeptide in the tumor cells.

In one aspect, the invention features a viral vector that includes a nucleic acid encoding a therapeutic polypeptide, wherein the nucleic acid is operably linked to a heterologous destabilizing element. Upon introduction of the vector into a target cell (e.g., a tumor cell), expression of the therapeutic polypeptide encoded by the nucleic acid is enhanced in the target cell relative to the expression of the therapeutic polypeptide in a non-target cell into which the vector has been introduced. The heterologous destabilizing element can be radiation responsive, responsive to inflammatory mediators (e.g., the 3′ UTR of the tumor necrosis factor alpha gene), stabilized in proliferating cells, responsive to activated RAS and elevated P-MAPK activity (e.g., 3′ UTR of the COX-2 gene), or responsive to hypoxic conditions (e.g., 3′ UTR of the vascular permeability factor/vascular endothelial growth factor gene or 3′ UTR of the urokinase plasminogen activator receptor gene).

In another aspect, the invention features a conditionally replication competent viral vector. The vector can include an essential gene operably linked to a heterologous destabilizing element, wherein upon introduction of the vector into a target cell (e.g., a tumor cell), expression of the essential gene product encoded by the essential gene is enhanced relative to the expression of the essential gene product in a non-target cell into which the viral vector has been introduced. The viral vector can be an adenoviral vector and the essential gene can be E1A. The viral vector also can be a vaccinia virus vector.

The invention also features a method of treating a patient having a tumor. As used herein, “treating a patient” refers to slowing of tumor growth, stopping tumor growth, reducing tumor size, or disappearance of tumor. The method includes administering to the patient a conditionally replication competent viral vector, or a conditionally replication competent virus containing a viral vector, wherein the viral vector contains an essential gene operably linked to a heterologous destabilizing element, whereby expression of the essential gene product encoded by the essential gene is enhanced in cells within the tumor relative to expression of the essential gene product in non-tumor cells into which the virus has been introduced. The virus can be an adenovirus and the essential gene can be the E1A gene. The virus can be a vaccinia virus. The heterologous destabilizing element can be radiation responsive, responsive to inflammatory mediators, stabilized in proliferating cells, responsive to activated RAS and elevated P-MAPK activity, or responsive to hypoxic conditions.

In yet another aspect, the invention features a method of treating a patient having a tumor. The method includes administering to the patient a viral vector, or a virus containing a vector, wherein the vector includes a nucleic acid encoding a therapeutic polypeptide operably linked to a heterologous destabilizing element, whereby expression of the therapeutic polypeptide is enhanced in cells within the tumor relative to expression of the therapeutic polypeptide in non-tumor cells into which the virus has been introduced.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a schematic of the construction of plasmids CMV-E1A and CMV-E1A-COX. The adenoviral E1A cDNA was PCR cloned into the expression plasmid pCR3.1 to generate CMV-E1A. A 469 bp fragment of the 3′UTR of the COX-2 gene (Dixon, D. A., et al. J Biol Chem 275, 11750-11757. (2000)) was cloned from genomic DNA by PCR and ligated downstream of the E1A gene to give CMV-E1A-COX. FIG. 1B is a Western blot indicating the growth of RIE-iRAS cells in 5 mM IPTG in culture leads to induotion of Ha-Ras^{Val12}. FIG. 1C is a graph of the increased growth rate of RIE-iRAS cells after induction of the Ha-Ras^{Val12} gene by 5 mM IPTG.

FIG. 2A and FIG. 2B are bar graphs that depict that the induction of the Ha-Ras^{Val12} gene in RIE-iRAS cells stabilizes E1A expression sufficiently to allow mobilization of a replication incompetent adenoviral vector. In FIG. 2A, 5×10⁵ RiE-Ras cells were plated in the presence or absence of IPTG (5 mM). 24 hours following transfection with 1.0 μg of CMV-E1A or CMV-E1A-COX DNA, the cells were infected with a replication-defective Ad-GFP (10 m.o.i.) vector. Spread of the GFP reporter gene through the culture was assayed with time using FACS analysis. Expression of GFP is shown at 72 hours following Ad-GFP infection. Control cells were transfected with an irrelevant plasmid and infected with Ad-GFP. Results shown are representative of four different experiments. In FIG. 2B, HT1080 cells were exposed to 72 hour supernatants following Ad-GFP infection of the RIE-iRAS cells transfected with either CMV-E1A or CMV-E1A-COX and treated +/−IPTG as shown. Infected HT1080 cells were analyzed by FACS to detect levels of Ad-GFP.

FIG. 3A and FIG. 3B depict that that the inhibition of Ha-Ras^{Val12}-induced P-MAPK activation by PD98059 blocks COX-2 3′UTR-mediated stabilization of E1A expression. FIG. 3A is a Western blot to determine levels of Ha-Ras^{Val12} and activated P-MAPK in RIE-iRas cells treated with IPTG in the presence of DMSO or PD98059 (50 μM) for 48 h. FIG. 3B represent fluorescence and FACs analysis measuring the mobilization of an Ad-GFP replication-incompetent vector through RIE-iRAS cells treated. RIE-iRAS cells were transfected with (i-iii) CMV-E1A or (iv-vi)CMV-E1A-COX (1.0 μg), infected with Ad-GFP at an m.o.i. of 10 and then treated with IPTG (ii and v) or IPTG and PD98059 (iii and vi). 72 hours later, the number of cells expressing GFP (a measure of the mobilization of the Ad-GFP by E1A expression) was measured as shown. vii) represents uninfected cells and viii) cells infected with 8×10⁵ pfu Ad-GFP.

FIG. 4A and FIG. 4B indicate that the replication of Ad-E1A-COX correlates with the P-MAPK status of tumor cell lines. RIE-iRAS cells grown in the presence or absence of IPTG to induce expression of the Ha-Ras^{Val12} oncogene were infected with Ad-E1A or Ad-E1A-COX viruses at an m.o.i. of 10. After 7 days, surviving cells were counted (FIG. 4A). FIG. 4B is a Western blot depicting the levels of P-MAPK activity in the cell lines used to assess the in vitro cytotoxicity of the Ad-E1A and Ad-E1A-COX viruses. Lanes 1-6, human lines: normal bronchial epithelial cells (BEAS); 2, fibrosarcoma, HT1080; 3, glioma U118; 4, glioma U87; 5 glioma U251; 6, colorectal HCT116. Lanes 7 and 8 rat intestinal epithelial RIE-iRAS cells grown in the absence (7) or presence (8) of IPTG to induce expression of Ha-Ras^{Val12}. Lane 9, 10 human prostatic LnCap and PC3 cells respectively. The range of tumor cell lines, characterized in FIG. 4B, were infected with Ad-E1A or Ad-E1A-COX viruses at an m.o.i. of 0.1. After 7 days, surviving cells were counted (FIG. 4C). FIG. 4D is a Northern blot analysis for expression of E1A mRNA of representative low P-MAPK (BEAS) or high P-MAPK (HCT116 and LnCap) cell lines infected with Ad-E1A (lanes 1, 3, 5) or Ad-E1A-COX (lanes 2, 4, 6) viruses as described in FIG. 4C.

FIG. 5 depicts that Ad-E1A-COX is selectively oncolytic to tumors expressing high levels of P-MAPK. FIG. 5A is a Western blot depicting the levels of P-MAPK activity in glioma cell lines (U118, U87 and U251). FIG. 5B is a Western blot depicting the levels of E1A expression in glioma cell lines (U87, U118 and U251) 15 hours following infection with Ad-E1A or Ad-E1A-COX viruses at an m.o.i. of 10. In FIG. 5C and FIG. 5D, U118 (C) or U251 (D) tumors were implanted subcutaneously in nude mice (10 mice per group) and allowed to develop to sizes of between 0.2-0.04 cm. These established tumors were injected directly with equal doses of Ad-E1A, Ad-E1A-COX (10⁸ pfu) or PBS, in a total volume of 100 μl and tumor growth was followed with time. The same experiments as described in FIGS. 5C and 5D were carried out with the U118 and U87 tumor lines. Experiments were terminated 60 days following virus injection when mean tumor sizes in all groups were measured. Results of different treatment groups, over different experiments, were expressed as a percentage of the mean size of the PBS injected control groups (FIG. 5E).

FIG. 6 indicates that the replication of Ad-E1A-COX cannot be detected following systemic administration. Mice (2 per group) were injected i.v with Ad-E1A or Ad-E1A-COX virus (10⁶ pfu/mouse). After 3 day, livers were recovered and used for preparation of cDNA, which was subsequently screened by PCR for levels of mRNA of E1A (FIG. 6A). Serum from these mice was recovered and plated in serial dilutions onto 293 cells. Presence of virus in the blood was assessed as cytopathic effect on the 293 cells and titer of circulating virus determined (FIG. 6B).

### DETAILED DESCRIPTION

In general, the invention features a method to control therapeutic gene expression by controlling mRNA stability. Viral vectors are used that incorporate destabilizing elements, in which the destabilizing element confers destabilizing activity to the mRNA to which it is operably linked, but whose actions are reversed under certain physiological conditions. Typically, a destabilization element contains at least a portion of a 3′ UTR that contains AU rich sequences. A wide variety of viral vectors can be used (e.g., adenovirus, vaccinia virus, herpes virus, reoviruses, Newcastle disease virus, retrovirus, adeno-associated virus, or Sindbis virus), including replication competent viral vectors (e.g., adenovirus, herpes virus, reoviruses, and Newcastle disease virus). For example, a conditionally replication competent adenoviral vector can be produced in which expression of the essential E1A gene is regulated by operably linking it to the 3′UTR of the COX-2 gene (a destabilizing element), allowing activated RAS/P-MAPK-specific stabilization of the EIA mRNA. Induction of activated RAS supports replication whereas matched cells in which activated RAS/P-MAPK is not expressed are very poor substrates for viral replication both in vitro and in vivo. This represents a replicating virus whose tumor selectivity is based upon control of gene expression at the level of mRNA stability.

Other destabilizing elements that confer destabilizing activity on their cognate mRNAs but whose actions are reversed under certain physiological conditions have been identified in a wide range of genes. Without being bound to a particular mechanism, genes regulated by this mechanism tend to be those induced when rapid changes of gene expression are required for cell proliferation or in response to inflammation. Examples include cytokines (Caput, D. et al. Proc Natl Acad Sci USA 83, 1670-1674. (1986); Shaw, G. & Kamen, R. Cell 46, 659-667. (1986); and Brook, M., et al.. FEBS Lett 483, 57-61. (2000)), cyclins (Maity, A., et al., Embo J 14, 603-609 (1995) and Maity, A., et al. Cell Growth Differ 8, 311-318. (1997)), other mediators of inflammatory reactions (Lindsten, T., et al., Science 244, 339-343 (1989)); Sheng, H., et al. Cancer Res 61, 2670-2675. (2001); and Claffey, K. P. et al. Mol Biol Cell 9, 469-481. (1998)), and proto-oncogenes (Doyle, G. A., et al. Cancer Res 60, 2756-2759 (2000); Bauer, S. R. et al. Oncogene 4, 615-623. (1989); and Chen, C. Y., et al. Mol Cell Biol 15, 5777-5788 (1995)).

One example of a protein which is both a mediator of inflammatory reactions and whose expression in various tumor types has been associated with poor prognosis is COX-2 (Cao, Y. & Prescott, S. M. J Cell Physiol 190, 279-286. (2002); Bakhle, Y. S. Br J Pharmacol 134, 1137-1150. (2001); and Turini, M. E. & DuBois, R. N. Annu Rev Med 53, 35-57 (2002). Expression of COX-2 is normally induced in cells by cytokines, growth factors and tumor promoters. Up-regulation of COX-2 is a downstream effect of RAS-mediated transformation (Sheng, H. et al. J Biol Chem 273, 22120-22127. (1998)). A large component of its up-regulation is mediated by selective stabilization of the mRNA of the COX-2 gene in RAS-transformed cells. mRNA stability has been shown to map to a region in the 3′UTR of the COX-mRNA (Sheng, H. et al. J Biol Chem 275, 6628-6635. (2000); and Dixon, D. A., et al., J Biol Chem 275, 11750-11757. (2000)). This mRNA stabilization was mediated in part through activation of the mitogen activated protein kinase P-MAPK pathway, which is a downstream effector of both RAS-, and EGF-receptor-, mediated intracellular signaling. Finally, the P-MAPK signaling cascade also is involved in preferential stabilization of other growth promoting mRNAs (Montero, L. & Nagamine, Y. Cancer Res 59, 5286-5293. (1999); and Gallouzi, I. E. et al. Mol Cell Biol 18, 3956-3965. (1998)) and proteins that link RAS-mediated signaling and RNA turnover have also been identified.

Other suitable examples of destabilizing elements include hypoxic responsive 3′UTR elements (e.g., at least a portion of the 3′ UTR of the vascular permeability factor/vascular endothelial growth factor gene or at least a portion of the 3′ UTR of the urokinase plasminogen activator receptor gene), radiation responsive elements, elements responsive to inflammatory mediators (e.g., at least a portion of the 3′ UTR of the tumor necrosis factor alpha gene), and 3′UTRs which mediate increased message stability in proliferating cells.

Viral vectors can incorporate other regulatory elements to confer multiple levels of specificity to the resultant virus. For example, tissue specific promoters can be used. Molecular features that target tumor cell specific mutations, such as loss of p53 or downstream effectors, also can be incorporated.

The invention will be further described in the following examples, which do not limit the scope of the invention described in the claims.

### EXAMPLES

### Experimental Protocol

Cell lines: The human tumor cell lines, HT1080 fibrosarcoma, HCT116 colorectal, U118, U87, U251 gliomas and LnCap, PC-3 prostate were obtained from the American Type Culture Collection (ATCC; Manassas, Va.) and were maintained as a monolayer in Dulbecco's modified Eagle's medium (DMEM, Life Technologies, Inc.) containing 10% fetal bovine serum. BEAS-2B (ATCC) is an immortalized normal human bronchial epithelial cell line. The RIE-iRas cell line with an inducible activated Ha-Ras^{Val12} cDNA was generated by using the LacSwitch eukaryotic expression system (Stratagene, La Jolla, Calif.) and was maintained in DMEM containing 400 μg/ml G418 (Life Technologies, Inc), 150 μg/ml hygromycin B (Invitrogen, Carlsbad, Calif.) and 10% FBS.

Plasmid construction: The 469 bp human COX-2 3UTR cDNA clone was isolated by reverse transcription-polymerase chain reaction (PCR) amplification using human COX-2 sequence-specific primers. PCR products were ligated into the TOPO TA-cloning vector (Invitrogen, Carlsbad, Calif.) and subsequently excised with Xho1. The DNA fragments were purified by agarose gel electrophoresis and extracted using Gene clean Kit (QIAGEN, Valencia, Calif.). DNA inserts were ligated into the unique Xho1 site of the pE1A-K2 vector (pE1A-K2-COX), located in the 3′-end of the adenovirus type 5 E1A gene. Cells were transiently transfected using Effectene transfection reagent (QIAGEN, Valencia, Calif.) according to the manufacturer's instructions.

Construction and production of recombinant adenovirus: The Ad-E1A-COX is an E1/E3 deleted, serotype 5 vector that contains the cytomegalovirus (CMV) immediate-early gene promoter-enhancer driving the adenovirus E1A cDNA (100 bp) which is fused with COX-2 3′ UTR (469 bp). This vector was constructed by using an AdEasy kit, according to the manufacture's protocol (Qbiogene, Calif.). Briefly, E1A-COX-2 gene was PCR cloned from plasmid pE1A-K2-COX and inserted into the transfer plasmid, pShuttle (AdEasy kit, Qbiogene, Calif.) by using the unique HindIII-EcoRV sites. The resulting plasmid (pShuttle-E1A-COX) was than linearized with Pme1 and co-transfected into E. coli strain BJ5183 together with pAdEasy-1 (Qbiogene, Calif.), the viral DNA plasmid. The recombinant adenoviral construct was then cleaved with PacI to expose its Inverted Terminal Repeats and transfected into 293A cells to produce viral particles. The viral clones were screened by PCR diagnosis of Hirt extracts. The selective vector clone was then plaque purified at least three times before it was used in experiments. For in vivo experiments, the virus was purified on cesium chloride gradient columns.

Immunoblot analysis for detection of proteins: Protein extracts were prepared at 11-14 h postinfection by lysis of infected cells with radioimmunoprecipitation assay buffer (10 mM Tris buffer [pH 7.4], 425 mM NaCl, 1% NP40, 1% deoxycholate, 0.1% SDS, 100 μl of protease inhibitor cocktail [Roche, Indianapolis, Ind.], 5 mM EGTA, 100 μM Na₃VO₄, 50 mM NaPyrophosphate, 50 mM NaF) and protein expression was determined by Western Blot after separation of 10 μg of cell lysate on 10% SDS-PAGE and transferred to a PVDF membrane (Millpore, Bedford, Mass.). The protein concentration was determined using a BCA protein assay with bovine serum albumin as a standard (Pierce, Rockford, Ill.). The detection of adenovirus E1A and H-RAS proteins were accomplished using rabbit polyclonal antibody (Santa Cruz Biotech., Santa Cruz, Calif.) against the target proteins. Inmunoreactive bands were visualized by enhanced chemiluminescence (Pierce, Rockford, Ill.). For the detection of Phospho-MAP kinase a monoclonal antibody from New England Biolabs Inc. was used. The MAP kinase specific inhibitor PD98059 was also purchased from New England Biolabs Inc.

Northern blot analysis: Total cellular mRNA was extracted by using a Rneasy kit (QIAGEN, Valencia, Calif.), according to the manufacture's protocol. The mRNA samples (10 μg/lane) were separated on formaldehyde-agarose gels and blotted onto nitrocellulose membranes. The blots were hybridized with cDNA probes labeled with [α-³²P] dCTP by random primer extension (Stratagene, La Jolla, Calif.). After hybridization and wash, the blots were subjected to autoradiography. 18S rRNA signals were used to determine integrity of RNA and equality of the loading.

In vivo studies: To establish subcutaneous tumors, 4-5 week old athymic nu/nu female mice (Harlan Sprague Dawley, Inc., Indianapolis, Ind.) were injected with 2×10⁶ tumor cells (U118, U87, U251). When the tumors measured 0.3 cm in diameter, 1×10⁸ pfu of wild type Ad-5 or Ad-E1A-COX were injected intratumorally in a 0.05 ml volume. Control tumors were injected with equal volume of PBS only. Animals were examined every other day and euthanized if tumor size reached 1.0×1.0 cm. An animal was scored as tumor-free when tumor size remained <0.2 cm.

For evaluation of the relative uptake of virus into liver after i.v. injection, athymic nu/nu female mice (Harlan Sprague Dawley, Inc., Indianapolis, Ind.) were given 1×10⁸ pfu of wild type Ad-5 or Ad-E1A-COX in single tail vein injection and euthanized at 24 and 72 h postinoculation. Liver, blood and spleens were excised, divided, and processed for viral titer, western blot or RT-PCR. To determine the virus titers, tissues were homogenized and freeze/thawed three times, centrifuged, and the virus titer in the supernatant was determined by a plaque assay using 293A cells.

### A Model for Induction of Activated RAS Expression In Vitro

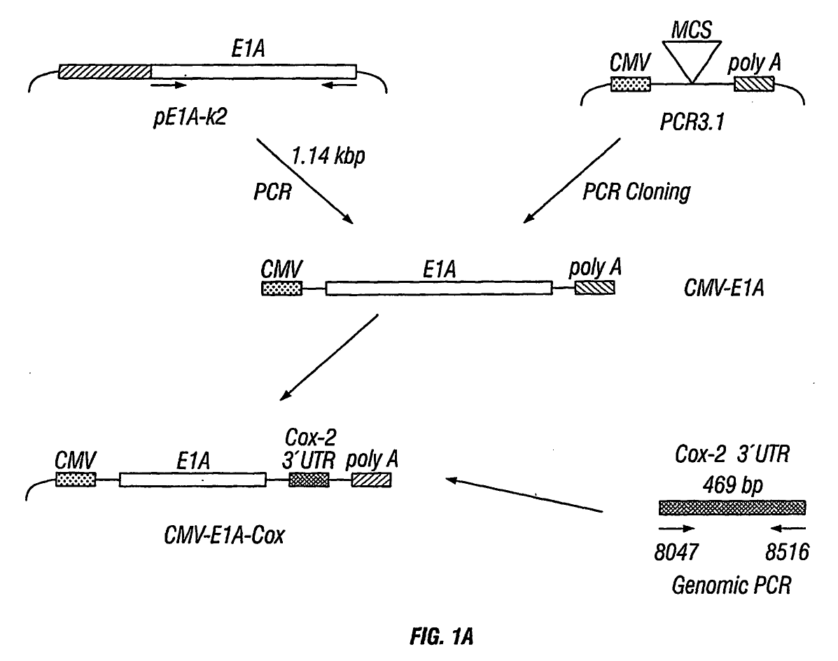

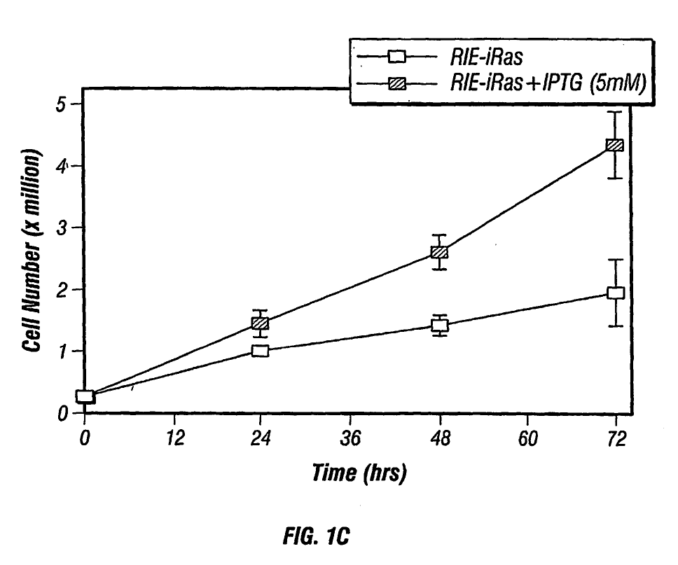

We hypothesized that it may be possible to exploit the RAS-mediated re-stabilization of mRNA linked to the COX-2 3′UTR to create an adenoviral vector that preferentially replicates in RAS-transformed cells. CMV-E1A contains the adenoviral E1A gene cloned downstream of the human CMV promoter (FIG. 1A). We then cloned 469 bp of the 3′UTR of the COX-2 gene downstream of the E1A gene in CMV-E1A to generate CMV-E1A-COX (FIG. 1A). In order to characterize the effects of the insertion of the 3′UTR on E1A expression, we used a model system in which exactly matched cell lines, differing only in the expression of an activated Ha-Ras^{Val-12} oncogene, could be compared. The rat intestinal epithelial cell line RIE-iRAS contains a stably integrated, IPTG-inducible activated Ha-Ras^{Val-12} cDNA. Addition of 5 mM IPTG to this line induces expression of Ha-Ras^{Val12} (FIG. 1B), which also leads to transformation of the cells as seen by morphological changes (data not shown), increased growth rate (FIG. 1C), and co-incident increased levels of expression of COX-2 (data not shown).

### E1A-COX Complements Adenoviral Replication in Trans Only in the Presence of Activated RAS Expression

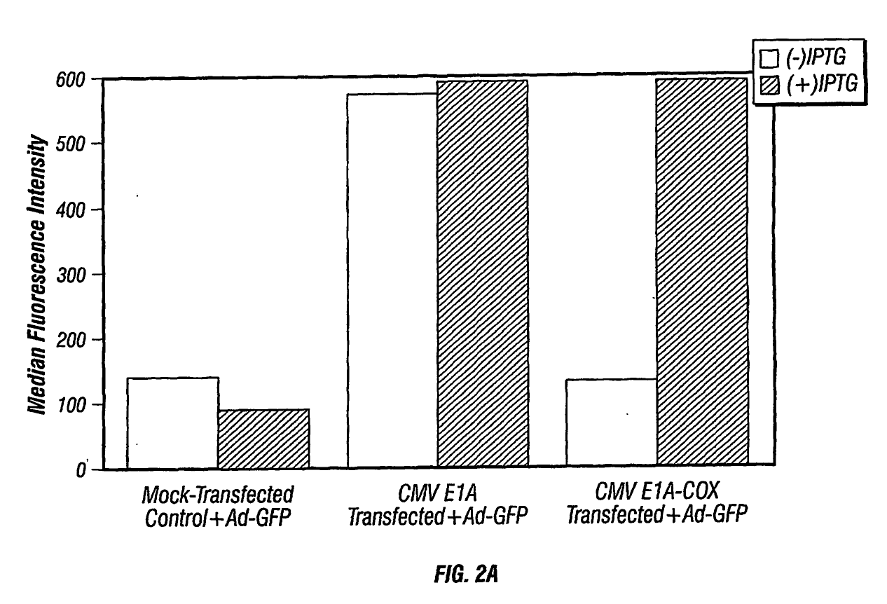

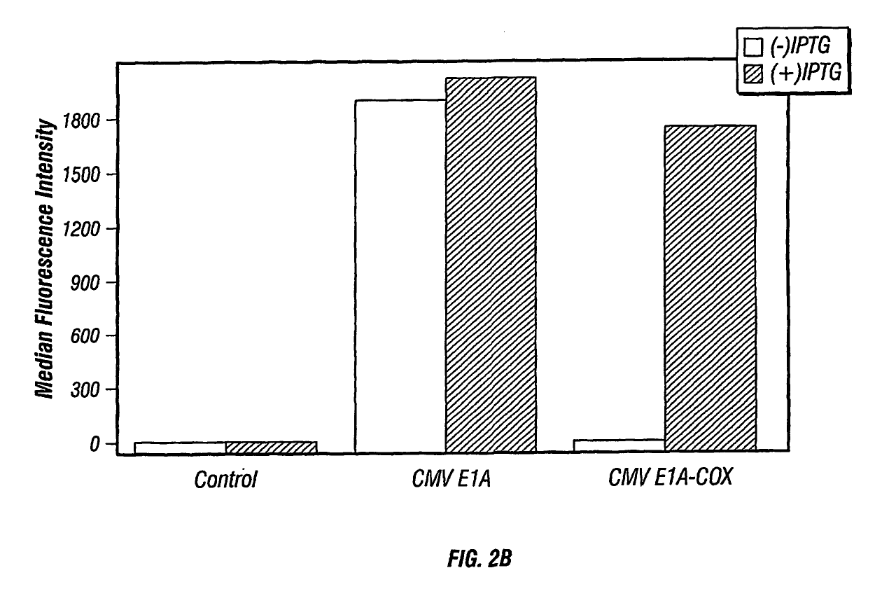

We investigated whether destabilization of the E1A mRNA by the COX-2 3′UTR in cells not transformed by RAS is functionally sufficient to block the mobilization of a replication-incompetent adenoviral vector. Although RIE-iRAS cells are of rodent origin, they are still able to support wild type adenoviral replication but at reduced levels compared to 293 cells (data not shown). RIE-iRAS cells transfected with CMV-E1A or CMV-E1A-COX were subsequently infected with an E1A-deleted replication incompetent adenoviral vector expressing GFP. E1A-expressing cells would be converted into transient adenoviral producer cells if they subsequently become infected with the Ad-GFP construct and would, therefore, mobilize the GFP reporter gene through the cell monolayer. FACS analysis of transfected/infected RIE-iRAS cells demonstrated that CMV-E1A supported considerable mobilization of the incoming Ad-GFP vector irrespective of the presence of IPTG (FIG. 2A). In contrast, CMV-E1A-COX was unable to mobilize the Ad-GFP vector to any significantly enhanced level compared to mock transfected cells unless cells were previously induced to express the Ha-Ras^{Val-12} oncogene by IPTG (FIG. 2A). To confirm that the mobilization of the GFP reporter gene was due to complementation in trans by the E1A proteins, supernatants were removed from the transfected/infected RIE-iRAS cultures and plated on HT1080 cells (FIG. 2B). FACS analysis of the infected HT1080 cells indicated that similar titers of Ad-GFP were present in the supernatants removed from CMV-E1A/Ad-GFP treated RIE-iRAS cells irrespective of the induction of Ha-Ras^{Val12}; however, there was only a significant titer of Ad-GFP released from CMV-E1A-COX/Ad-GFP-transduced RIE-iRAS cells if these cells were treated with IPTG to induce expression of Ha-Ras^{Val-12} (FIG. 2B). No cytopathic effect was observed in the infected HT1080 cells, indicating that the virus released from the RIE-iRAS cells was replication incompetent and derived from complementation of the Ad-GFP vector by the E1A proteins.

### COX-2 3′UTR-Mediated E1A Stabilization in Ha-RAS^{val-12} Transformed Cells is Dependent upon the MAP Kinase Pathway

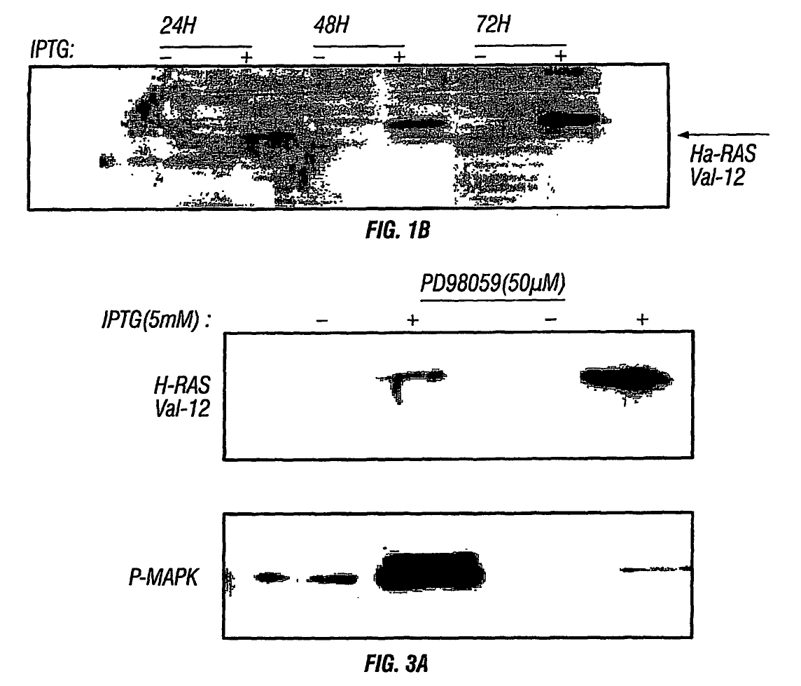

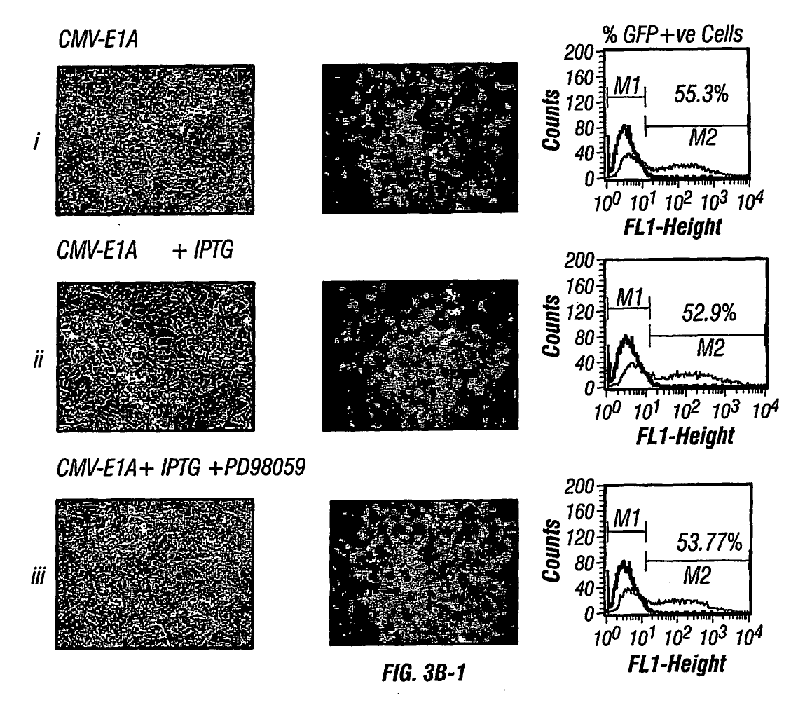

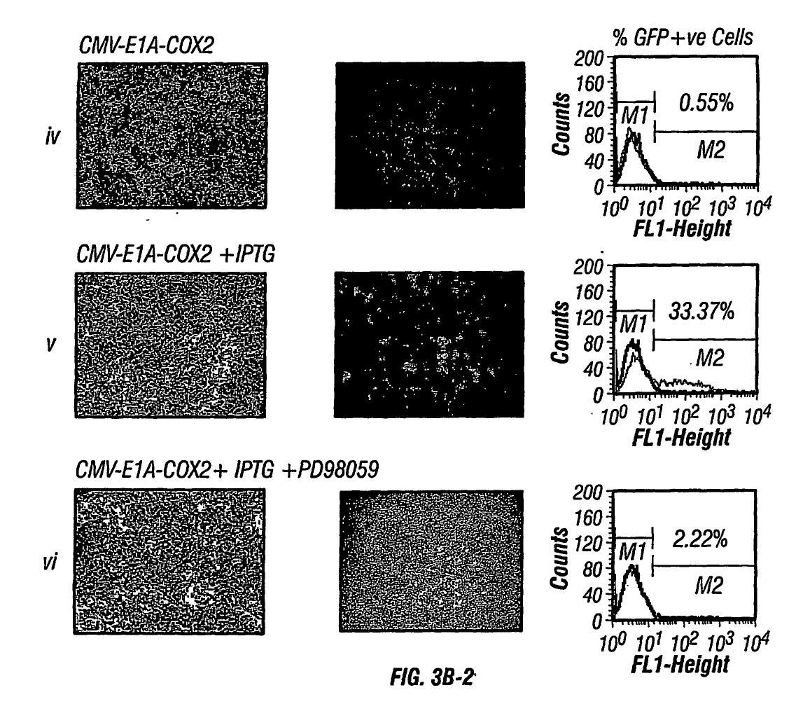

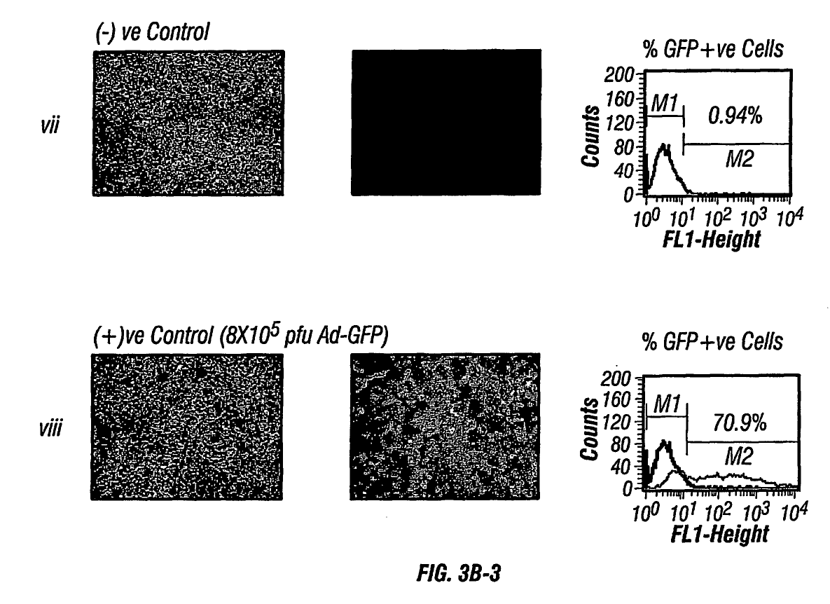

Inhibition of the P-MAPK pathway has been reported to block RAS-mediated induction of COX-2 expression. Therefore, we investigated whether the effects we observed with Ha-Ras^{Val-12}-mediated control of E1A-COX expression operate through the P-MAPK signaling pathway. RIE-IRAS cells express minimal levels of P-MAPK in the absence of Ha-Ras^{Val-12} induction (FIG. 3A). However, expression of the activated oncogene, even at relatively low levels, induces high levels of P-MAPK protein (FIG. 3A). PD98059, an inhibitor of P-MAPK activity, effectively blocked P-MAPK expression in RIE-iRAS cells even when the cells were induced to express high levels of Ha-Ras^{Val-12} (FIG. 3A). Therefore, we used PD98059 inhibition to demonstrate that the stabilization of E1A expression from CMV-E1A-COX is also dependent upon the P-MAPK pathway. Mobilization of the Ad-GFP adenoviral vector through RIE-iRAS cultures by transfection of CMV-ELA occurred at high levels in the absence (3B i) or presence (3B ii) of IPTG and PD98059 (3B iii) (FIG. 3B). As before, transfection of CMV-E1A-COX required IPTG induction of Ha-Ras^{Val-12} to be effective in vector mobilization (FIG. 3B iv, v). However, inhibition of P-MAPK activity by PD98059 greatly reduced the ability of CMV-E1A-COX to support Ad-GFP mobilization even in the presence of high levels of expression of Ha-Ras^{Val-12} induced by IPTG (FIG. 3B vi).

### E1A Expression Can be Destabilized Within an Adenoviral Genome by the COX-2 3′UTR and Re-Stabilized in the Presence of Activated RAS and High Levels of P-MAPK

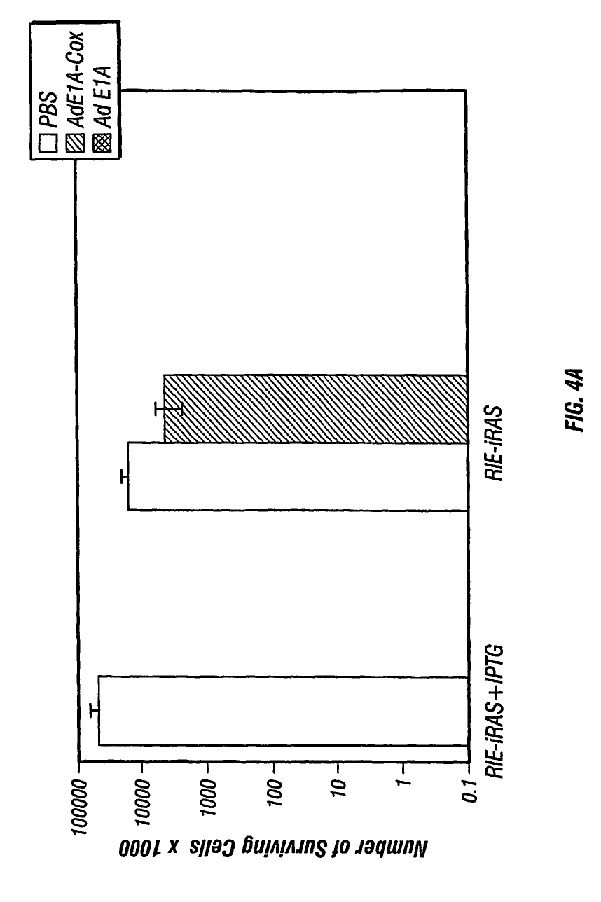

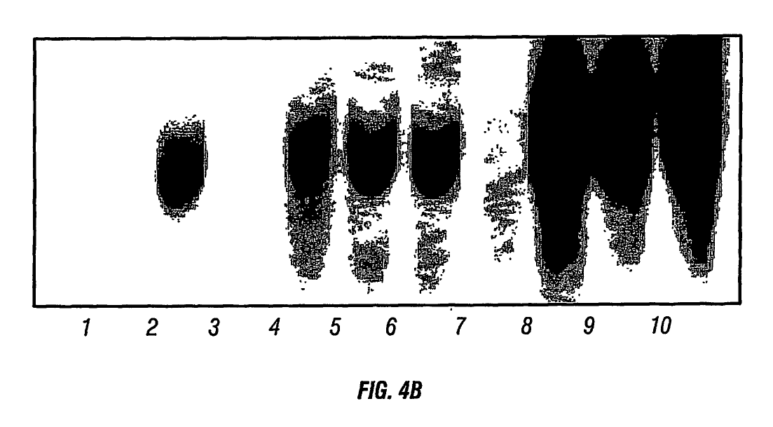

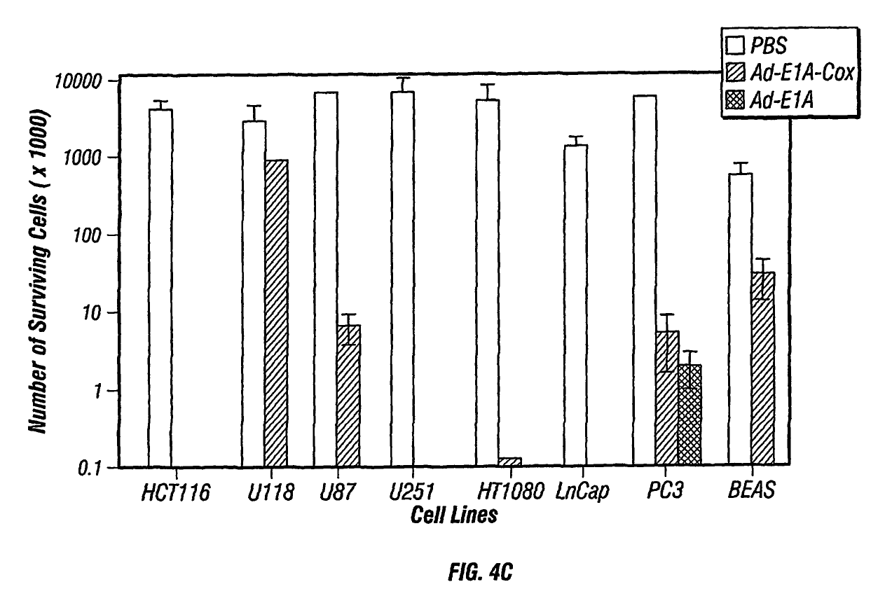

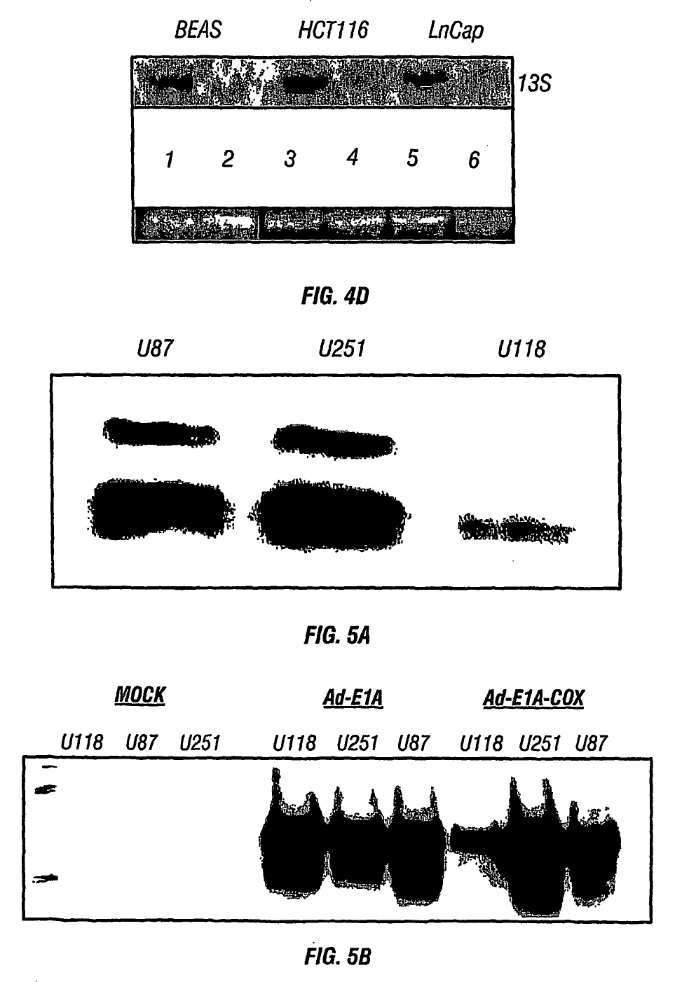

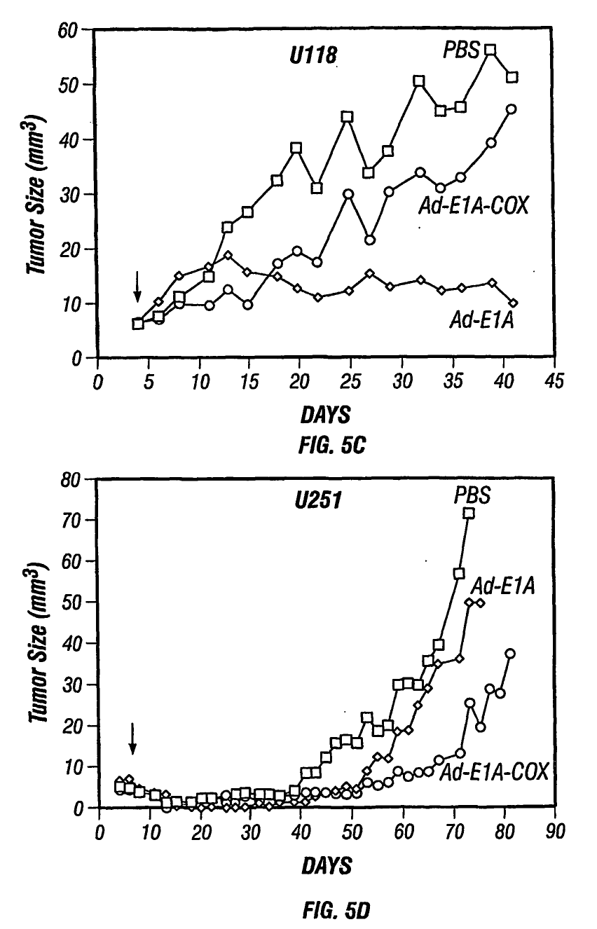

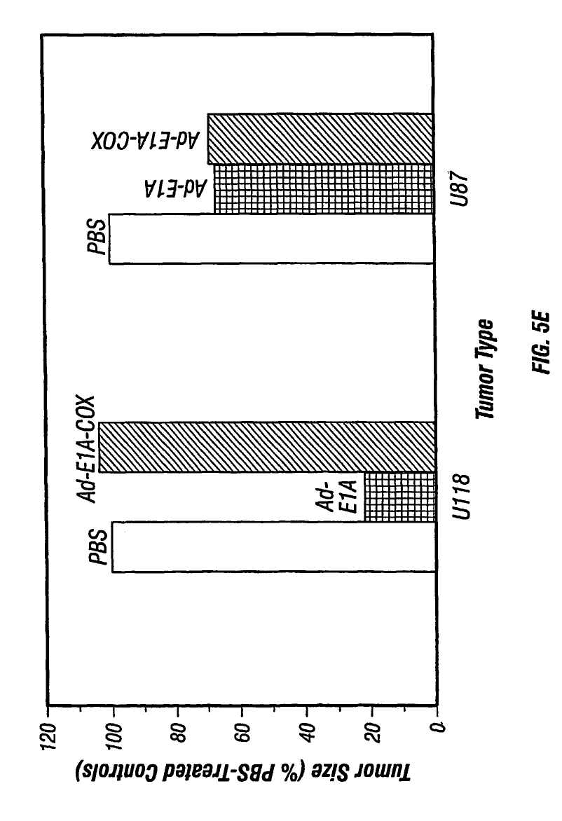

We incorporated the E1A and E1A-COX cassettes into E1A-deleted adenoviral genomes. Viruses recovered from transfection of 293 cells were purified and used to infect RIE-iRAS cells in the presence or absence of IPTG. Replication of Ad-E1A-COX in RIE-IRAS cells was heavily dependent upon IPTG induction of Ha-Ras^{Val-12} (FIG. 4A). In addition to the cytotoxicity assay described in FIG. 4A, we also assayed replication of the Ad-E1A-COX virus directly in infected RIE-IRAS cells (+/−) IPTG as described in Chong, H., et al.. Mol Ther 5, 195-203. (2002). Ad-E1A-cox-infected RIE-IRAS cells in the presence of IPTG consistently produced in excess of 3 logs more virus (10⁵ plaques per 10⁵ lysed infected cells) per cell than the same cultures in the absence of IPTG induction (10² plaques per 10⁵ lysed infected cells). We also wanted to confirm that the effects we observed in the model RIE-IRAS system were applicable to human cell lines with different levels of RAS or P-MAPK activity. Therefore, the levels of P-MAPK in several different human cell lines were measured by Western blot analysis (FIG. 4B). Of these lines, uninduced RIE-IRAS, U118 glioma and the normal epithelial BEAS cell lines expressed low or undetectable levels of P-MAPK. The remainder expressed moderate (HT1080, U87, U251 and HCT116) or high (IPTG-induced RIE-IRAS, LnCap and PC3) levels of P-MAPK (FIG. 4B). Therefore, these lines were infected with the Ad-E1A or Ad-E1A-COX viruses (at a lower m.o.i. than with the RIE-IRAS line because of the improved ability of human lines to support adenoviral replication). 7 days following infection with an m.o.i. of 0.1, surviving cells were counted (FIG. 4C). The wild type E1A gene supported ongoing viral replication that caused lysis and lclling of every cell line although the efficacy of the wild type virus was reduced in the human prostatic line PC3 (FIG. 4C). In contrast, the replication of Ad-E1A-COX was much more heavily dependent upon the cell line; in general, oncolysis correlated very closely with the line's P-MAPK activity status. Thus, cultures of normal bronchial epithelial cells (BEAS) were completely eradicated by Ad-E1A virus infection (FIG. 4C) but Ad-E1A-COX was significantly less toxic to these cells (which are very sensitive to adenoviral infection) but which have no detectable P-MAPK activity by Western Blot. Ad-E1A-COX also replicated only very poorly relative to the wild type Ad-E1A in the U118 (glioma) (FIG. 4C) and uninduced RIE-iRAS cells lines (FIG. 4A). We did observe some killing of U118 cells by Ad-E1A-COX due to the fact these cells express low, but still detectable, levels of P-MAPK (see, for example, FIG. 5A below). Infection of the U87 glioma line (moderate P-MAPK activity) with Ad-E1A-COX was effective at killing these cells although not as well as the wild type virus. In contrast, one other glioma cell lines U251, the fibrosarcoma HT1080 and colorectal HCT-116 cell lines and two prostate cell lines LnCap and PC3 were as good substrates for replication of Ad-E1A-COX as for Ad-E1A and all 5 lines express elevated levels of P-MAPK. As for the RIE-iRAS cells in FIG. 4A above, viral burst assays from the infected human cell lines confirmed the cytotoxicity data in that P-MAPK expressing cells produced in general 3-4 or 2 logs more virus per infected cell in a replication assay than the BEAS or U118 cell lines respectively. In addition, Northern blot analysis confirmed a direct correlation between the levels of oncolysis of different cell lines, the levels of P-MAPK activity and the expression of steady state levels of E1A mRNA species at early time points following infection with Ad-E1A or Ad-E1A-COX (FIG. 4D).

### Ad-E1A-COX is Oncolytic in vivo Against Human Tumors Expressing High Levels of P-MAPK Activity

Although the RIE-iRAS cell line grew in nude mice, we observed induction of Ha-Ras^{Val-12} within all of the tumors in vivo, irrespective of whether IPTG was administered. Therefore, to test the selectivity of the Ad-E1A-COX virus, we used tumor lines that are closely matched histologically, but which differ in levels of P-MAPK activity. For this reason, we used the glioma cell lines U118 (P-MAPK low) and U87 or U251 (P-MAPK moderate/high) (FIG. 5A) to test the in vivo efficacy and selectivity of the Ad-E1A-COX virus. Western Blotting confirmed in vitro that the level of E1A expression supported by Ad-E1A and Ad-E1A-COX infection (FIG. 5B) reflects very closely the P-MAPK activity of these lines (U118<<U87<U251) (FIG. 5A) data which was confirmed at the RNA level by Northern Blotting (data not shown).

There was a significant difference in the growth rate of established U118 tumors following a single intratumoral injection of wild type Ad-E1A compared to tumors injected with PBS (p<0.001) (FIG. 5C). However, injection with Ad-E1A-COX virus gave no statistically significant difference compared to the PBS injected control (FIG. 5C) in U118 (low P-MAPK activity) tumors. In the U251 model, the oncolytic effects of a single intratumoral injection of wild type adenovirus were reduced compared to those in the U118 model (FIG. 5D), but, consistent with the high levels of P-MAPK activity in this tumor (FIG. 5A), the Ad-E1A-COX virus either matched, or in the example shown in FIG. 5D, exceeded the efficacy of the wild type virus (FIG. 5D). These findings were also confirmed in the second P-MAPK high glioma model, U87. Thus, combining the results of several experiments, injection of wild type Ad-E1A virus was effective at reducing the size of U118 tumors (>75% reduction in final tumor size relative to PBS injected control tumors). This therapeutic effect was less effective in the U87 model (˜30% reduction with respect to PBS injected tumors) (FIG. 5E). Ad-E1A-COX was, however, as effective as wild type adenovirus when used to treat U87 tumors (moderate/high P-MAPK activity) but had no significant effect on treatment of subcutaneous U118 tumors (FIG. 5E) (low levels of P-MAPK activity and de-stabilized E1A expression as shown in FIG. 5B). The fact that Ad-E1A-COX was even more effective than wild type virus in the U251 model (FIG. 5D), but that Ad-E1A-COX was only similar to wild type virus in efficacy in the U87 model (FIG. 5E) is consistent with the observation that U251 tumors express somewhat higher levels of P-MAPK than U87 tumors (FIGS. 4B and 5A) and accordingly support higher levels of adenoviral replication (FIGS. 4C and 5B). Taken together, these in vivo results are consistent with the in vitro data demonstrating a strong correlation between the P-MAPK status of a tumor and its ability to support replication of the Ad-E1A-COX virus.

### AdE1A-COX Shows Reduced E1A Expression in Normal Tissues Following Systemic Administration

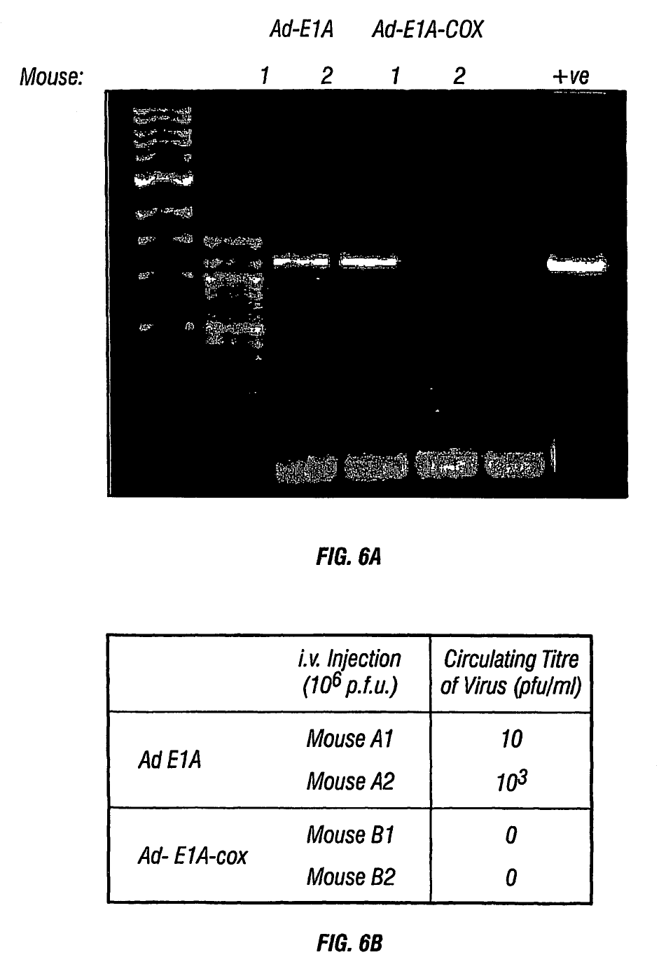

Given the particular sensitivity of the liver as a potential site of toxicity following therapy with adenoviral vectors, we tested whether the selectivity of Ad-E1A-COX for non-transformed cells was also maintained in normal liver in vivo. Mice were injected intravenously with either wild type Ad-E1A or Ad-E1A-COX virus (10⁶ pfu per mouse) in order to infect normal hepatocytes and other tissues. Three days later, livers were removed from the animals and assayed for expression of E1A mRNA by rtPCR. Hepatic expression of E1A could be detected following infection with Ad-E1A virus in both treated mice (FIG. 6A). However, the presence of the COX-2 3′UTR was sufficient to lower levels of expression of E1A mRNA to below detectable levels in both mice injected with Ad-E1A-COX virus (FIG. 6A). Serum was also collected from the treated mice and tested for the presence of replicating virus. Serial dilutions of samples plated onto 293 cells indicated that mice treated with Ad-E1A had very low, but detectable titers of circulating virus (FIG. 6B), presumably as a result of low level replication in the liver or elsewhere. In contrast, no detectable virus could be recovered from either mouse treated i.v. with Ad-E1A-COX virus. Therefore, the presence of the COX-2 3′UTR reduces significantly levels of E1A expression and viral replication in normal liver tissue.

In summary, the CMV-E1A plasmid construct generated functional E1A proteins that could complement, in trans, the mobilization of a replication incompetent Ad-GFP adenoviral vector in any cell irrespective of its Ha-Ras^{Val-12} status. However, the CMV-E1A-COX plasmid led to appreciable functional levels of E1A expression only in the presence of an activated Ha-Ras^{Val-12} protein in the RIE-IRAS model system. Moreover, using inhibition studies, we confirmed that COX-2 3′UTR-mediated stabilization of E1A expression in this system occurs through the P-MAPK signaling pathway which is up-regulated in the presence of an activated Ha-Ras^{Val-12} oncogene.

Using a panel of cell lines in which P-MAPK activity was characterized through Western Blotting, we showed that the Ad-E1A-COX virus is preferentially oncolytic in vitro in human tumor cells with high levels of P-MAPK activity. In vivo, the Ad-E1A-COX virus was at least as effective oncolytically as wild type virus in high P-MAPK expressing tumors (U87 and U251), but generated no significant therapeutic effects in low P-MAPK expressing tumors (U118). We also demonstrated that the selectivity of the COX-2 3′UTR is strictly maintained in vivo and acts to diminish adenoviral replication in normal liver tissue. Adenoviral replication in murine cells is greatly reduced compared to human cells but in vivo toxicity studies have shown that intravenous administration of adenoviral vectors results mostly in hepatocyte transduction. Following intravenous injection of adenovirus, virus could be detected in the blood of mice receiving Ad-E1A but not Ad-E1A-COX virus. Moreover, the livers of injected mice expressed appreciable levels of E1A from the Ad-E1A virus. In contrast, no expression of E1A could be detected at the level of mRNA in normal livers of mice following similar injections of Ad-E1A-COX. Given the particular sensitivity of the liver as a potential site of toxicity following therapy with adenoviral vectors, these data show that the presence of the COX-2 3′UTR is sufficient to reduce significantly levels of E1A expression in normal liver tissue. This would be expected to translate into significantly reduced levels of toxicity should such vectors become disseminated through the circulation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A viral vector comprising a nucleic acid encoding a therapeutic polypeptide, wherein said nucleic acid is operably linked to a heterologous destabilizing element, wherein upon introduction of said vector into a target cell, expression of said therapeutic polypeptide encoded by said nucleic acid is enhanced in said target cell relative to the expression of said therapeutic polypeptide in a non-target cell into which said vector has been introduced.

2. The vector of claim 1, wherein said target cell is a tumor cell.

3. The vector of claim 1, wherein said heterologous destabilizing element is radiation responsive.

4. The vector of claim 1, wherein said heterologous destabilizing element is responsive to inflammatory mediators.

5. The vector of claim 4, wherein said heterologous destabilizing element is the 3′ untranslated region of the tumor necrosis factor alpha gene.

6. The vector of claim 1, wherein said heterologous destabilizing element is stabilized in proliferating cells.

7. The vector of claim 1, wherein said heterologous destabilizing element is responsive to activated RAS and elevated P-MAPK activity.

8. The vector of claim 7, wherein said heterologous destabilizing element is the 3′ untranslated region of the cyclooxygenase 2 gene.

9. The vector of claim 1, wherein said heterologous destabilizing element is responsive to hypoxic conditions.

10. The vector of claim 9, wherein said heterologous destabilizing element is the 3′ untranslated region of the vascular permeability factor/vascular endothelial growth factor gene.

11. The vector of claim 9, wherein said heterologous destabilizing element is the 3′ untranslated region of the urokinase plasminogen activator receptor gene.

12. A conditionally replication competent viral vector, said vector comprising an essential gene operably linked to a heterologous destabilizing element, wherein upon introduction of said vector into a target cell, expression of the essential gene product encoded by said essential gene is enhanced in said target cell relative to the expression of the essential gene product in a non-target cell into which said viral vector has been introduced.

13. The viral vector of claim 12, wherein said target cell is a tumor cell.

14. The viral vector of claim 12, wherein said viral vector is an adenoviral vector.

15. The viral vector of claim 14, wherein said essential gene is E1A.

16. The viral vector of claim 12, wherein said viral vector is a vaccinia virus vector.

17. A method of treating a patient having a tumor, said method comprising administering to said patient a conditionally replication competent viral vector, said vector comprising an essential gene operably linked to a heterologous destabilizing element, whereby expression of the essential gene product encoded by said essential gene is enhanced in cells within the tumor relative to expression of the essential gene product in non-tumor cells into which the virus has been introduced.

18. The method of claim 17, wherein said virus is an adenovirus.

19. The method of claim 18, wherein said essential gene is the E1A gene.

20. The method of claim 17, wherein said virus is a vaccinia virus.

21. The method of claim 17, wherein said heterologous destabilizing element is radiation responsive, responsive to inflammatory mediators, stabilized in proliferating cells, responsive to activated RAS and elevated P-MAPK activity, or responsive to hypoxic conditions.

22. A method of treating a patient having a tumor, said method comprising administering to said patient a viral vector, said vector comprising a nucleic acid encoding a therapeutic polypeptide operably linked to a heterologous destabilizing element, whereby expression of said therapeutic polypeptide is enhanced in cells within said tumor relative to expression of said therapeutic polypeptide in non-tumor cells into which said virus has been introduced.
